(19) United States
(12) Patent Application Publication
(10) Pub. No.: US 2005/0070953 A1
(43) Pub. Date: Mar. 31, 2005

(54) Medical device with flexible distal end loop and related methods of use

(76) Inventor: James W. Riley, Bloomington, IN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/942,086

(22) Filed: Sep. 16, 2004

Related U.S. Application Data
(60) Provisional application No. 60/503,994, filed on Sep. 18, 2003.

(51) Int. Cl. A61M 29/00

(52) U.S. Cl. 606/200

(57) ABSTRACT

A device and related methods of use are provided for the capture and removal of various unwanted objects present within the body's anatomical lumens. In an embodiment of the present disclosure, the device includes a sheath; an actuation handle; an end-effector loop connected to the actuation handle and retractable within and extendable from a lumen of the sheath; and a connecting member disposed within the sheath for deflecting the end-effector loop relative to the longitudinal axis of the device. The end-effector loop includes an outer periphery that defines a space that may include a webbing.

### CROSS-REFERENCE TO RELATED APPLICATION

This patent application claims the benefits of priority under 35 U.S.C. §§119, 120 to U.S. Provisional Patent Application No. 60/503,994, entitled MEDICAL DEVICE WITH FLEXIBLE DISTAL END LOOP, filed on Sep. 18, 2003, the entirety of which is incorporated herein by reference.

### DESCRIPTION OF THE INVENTION

1. Field of the Invention

Embodiments of this invention relate generally to the field of medical devices and procedures for engaging material in a body. More particularly, embodiments of the instant invention relate to retrieval assemblies, such as baskets, graspers, forceps, or screens, for holding and/or entrapping material, such as a stone, in a body tract.

2. Background of the Invention

As known in the art, various specialized medical devices, such as graspers, balloons, and baskets, can be used to entrap an object, such as a stone, and drag it through an anatomical lumen to remove it. None of the existing devices can address all of the requirements of some procedures, for example, kidney stone removal procedures. Thus, a device that encompasses the functionality of graspers, balloons, and baskets is desirable.

### SUMMARY OF THE INVENTION

Embodiments of the invention provide a medical device and procedure for engaging material within a body. Methods in accordance with various embodiments of the present invention involve using the apparatus to grasp, move, remove, loosen, occlude, release, and/or sweep material in a body tract of a patient.

In accordance with an aspect of the present disclosure, a medical device may include: a sheath having a proximal end, a distal end, and a lumen extending longitudinally between the proximal and distal ends; an actuation handle disposed at the proximal end of the sheath; an end-effector loop adjacent a distal end of the sheath and operably connected to the actuation handle for actuation of the end-effector loop between a deployed position and an undeployed position; and a connecting member also disposed within the lumen of the sheath and connected to the actuation handle and the end-effector loop, wherein actuation of the connecting member deflects the end-effector loop when the end-effector loop is in the deployed position.

Various embodiments of the invention may include one or more of the following aspects: the deployed position is an expanded position and the undeployed position is a collapsed position; the end-effector loop is in the collapsed position when retracted into the sheath; the end-effector loop is extendable from and retractable within the lumen of the sheath; actuation of the connecting member deflects the end-effector loop when the end-effector loop is extended from the lumen of the sheath; an elongate actuation member connects the end-effector loop to the actuation handle for actuation of the end-effector loop; the elongate actuation member attaches to a proximal end of the end-effector loop, and the connecting member attaches to a distal end of the end-effector loop; the elongate actuation member is selected from a group comprising a wire and a cable; the connecting member moves within the lumen of the sheath; the end-effector loop comprises an outer periphery that defines a space including webbing; the webbing includes a plurality of wires; the webbing defines a plurality of spaces between the wires; the end-effector loop comprises an outer periphery that defines a space including a plurality of wires; actuation of the connecting member deflects the end-effector loop up to 180 degrees.

Another aspect of the present disclosure includes a method of entrapping an object within a body. The method includes inserting a medical device into a body lumen, the medical device having a sheath having a proximal end, a distal end, and a lumen extending longitudinally between the proximal and distal ends, an actuation handle disposed at the proximal end of the sheath, an end-effector loop adjacent a distal end of the sheath and operably connected to the actuation handle, and a connecting member disposed within the lumen of the sheath and connected to the actuation handle and the end-effector loop. The method further includes advancing the medical device to a desired body location within a body lumen, extending the end-effector loop distally relative to the sheath to deploy the end-effector loop into a deployed position, with the end-effector loop in the deployed position, actuating the connecting member to deflect the end-effector loop, and entrapping the object with the end-effector loop.

Various embodiments of the invention may include one or more of the following aspects: the step of removing the medical device and entrapped object from the body; the step of deploying the end-effector loop includes expanding the end-effector loop; an elongate actuation member operably connects the end-effector loop to the actuation handle; the elongate actuation member attaches to a proximal end of the end-effector loop, and the connecting member attaches to a distal end of the end-effector loop; the elongate actuation member is selected from a group comprising a wire and a cable; the connecting member moves within the lumen of the sheath; the end-effector loop comprises an outer periphery that defines a space including webbing; the webbing includes a plurality of wires; the webbing defines a plurality of spaces between the wires; the end-effector loop comprises an outer periphery that defines a space including a plurality of wires; the step of actuating the connecting member deflects the end-effector loop up to 180 degrees; the end-effector loop is extended distally relative to the sheath from a collapsed position; the step of prying an object from an embedded position within a body; the step of prying an object from an embedded position within a body is performed prior to entrapping the object with the end-effector loop; the end-effector loop is in the collapsed position when retracted into the lumen of the sheath.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate one embodiment of the invention and together with the description, serve to explain the principles of the invention.

FIG. 1 is a schematic representation of a medical device in an expanded and extended position according to embodiments of the present invention.

FIG. 2 is a schematic representation of a medical device in a retracted and collapsed position according to embodiments of the present invention.

FIG. 3 is a diagram illustrating dimensions of a medical device in an expanded and extended position according to various embodiments of the present invention.

FIG. 4 is a diagram illustrating a portion of a medical device in an expanded and extended position within an anatomical tract according to embodiments of the present invention.

FIG. 5 is a diagram illustrating the use of a portion of a medical device, according to embodiments of the present invention, to grasp material within an anatomical tract.

FIG. 6 is a diagram illustrating the use of a portion of a medical device, according to embodiments of the present invention, to sweep an anatomical tract occluded with material.

FIG. 7 is a diagram illustrating the use of a portion of a medical device, according to embodiments of the present invention, to pry impacted material from an anatomical tract occluded with material.

### DESCRIPTION OF THE EMBODIMENTS

Reference will now be made in detail to the present exemplary embodiments of the invention, an example of which is illustrated in the accompanying drawings. Wherever possible, the same reference numbers will be used throughout the drawings to refer to the same or like parts.

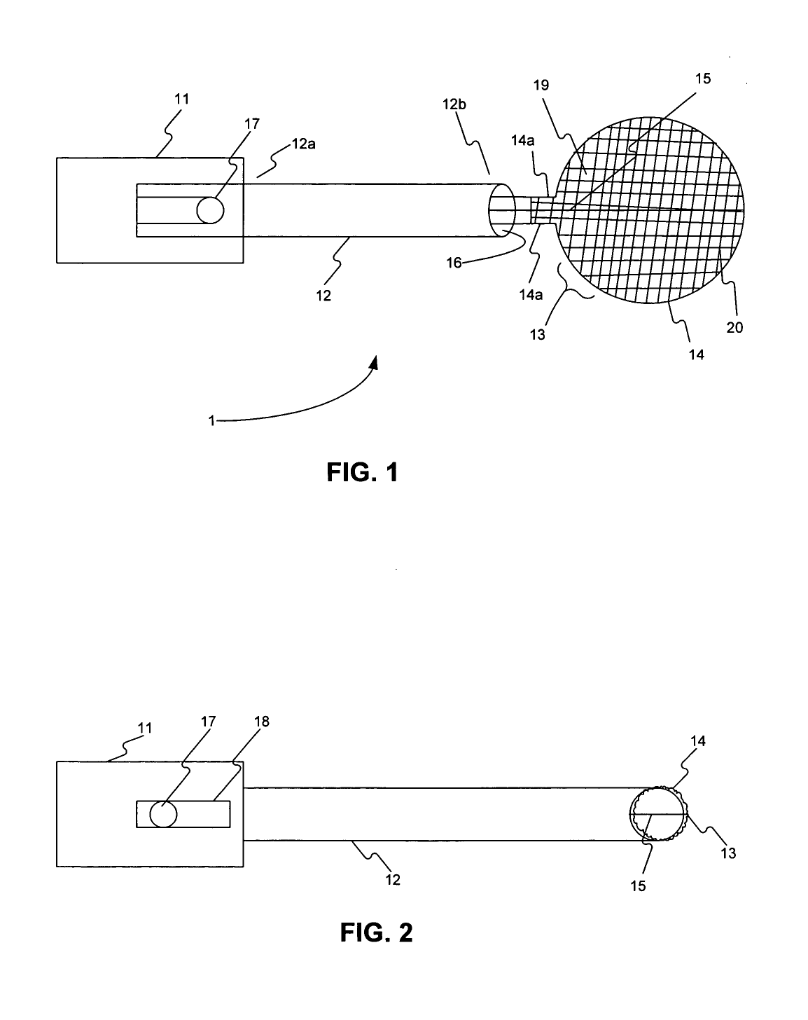

FIGS. 1-7 depict certain configurations of an exemplary embodiment of a medical device 1 in accordance with the present invention. The medical device 1 includes an actuation handle 11, a sheath 12 having a longitudinally disposed lumen 16, an end-effector loop 14 disposed relative to the distal end of the sheath 12, and at least one connecting member 15 disposed between the end-effector loop 14 and the actuation handle 11. As used in this disclosure, “distal” refers to a position or direction furthest from a user of the device and “proximal” refers to a position or direction opposite “distal” and closest to the user.

As shown in FIG. 1, the sheath 12 includes a proximal end 12a and a distal end 12b, and may be any suitable sheath or catheter known in the art. Sheath 12 may be fabricated by any known process such as, for example, extrusion. In addition, sheath 12 may be made from any suitable material. Such materials may include, but are not limited to, Teflon®, polyimide, and/or stainless steel. Sheath 12 may have any desired cross-sectional shape and/or configuration. For example, sheath 12 may have a substantially circular cross-section. Sheath 12 may also have one or more cross-sectional shapes and/or configurations along its length, and may be any desired dimension suitable for deployment within a desired body lumen. For example, sheath 12 may have dimensions adapted for placement in the human urinary tract having a particular size. The overall length and diameter of the sheath 12 may vary depending on application. For example, a relatively long sheath 12 may be advantageous for retrieving stones or other objects deep within the body of a patient. Furthermore, sheath 12 may also be flexible along at least a portion of its length so that it may conform to the contours of an existing anatomical tract or lumen.

Sheath 12 may further include at least one lumen 16 extending therethrough. In some embodiments, lumen 16 may be defined as an internal passageway with an entrance and an exit, and may be formed by any suitable process such as, for example, extrusion. Alternatively, the sheath 12 may include a plurality of lumens (not shown) that may or may not differ in size. In such embodiments, the plurality of lumens may provide passageways useful in delivering, or removing, for example, medical devices, fluids, and/or other structures to or from a treatment site. The lumen 16 provided within sheath 12 may have any cross-sectional shape, dimension, and/or configuration. For example, in some embodiments lumen 16 may have a substantially circular cross-section.

End-effector loop 14 may extend within lumen 16 of sheath 12, and may be disposed at a distal end of sheath 12 for longitudinal movement relative to the sheath 12. The end-effector loop 14 may have any desired shape and/or configuration. For example, the end-effector loop 14 may have a substantially circular shape. The end-effector loop 14 may include an outer periphery made from any suitable material. Such materials may include, but are not limited to, metals or metal compounds, such as stainless steel or nitinol. For example, end-effector loop 14 may be made from a wire. The wire may have any desired cross-sectional shape and or configuration. For example, the wire may have a round cross-section. The end-effector loop 14 may also have one or more shapes and/or configurations along its length, and any desired dimensions suitable for deployment in a desired body lumen. For example, end-effector loop 14 may have dimensions adapted for placement in the human urinary tract having a particular size. For instance, the end-effector loop 14, in an expanded state, may have an outer periphery that is substantially the same size as the inner periphery of the human urinary tract having a particular size. In addition, the end-effector loop 14 may have a configuration or dimensions, or have material properties, that permit longitudinal movement of the end-effector loop 14 relative to the sheath 12, and extension and retraction into and out of the sheath 12. The overall length D and diameter A of the end-effector loop 14 may vary depending on the application. For example, a relatively small diameter A may be advantageous for retrieving stones or other objects from restricted passageways within the human urinary tract. The end-effector loop 14 may also be collapsible so that it may be more easily advanced through body lumens in its collapsed state, and, when desired, self-expandable for deployment.

In the embodiment shown in FIG. 1, the space defined by the end-effector loop 14 is at least partly enclosed with a webbing 13. The end-effector loop 14 in various embodiments may be covered or coated with the webbing 13. The webbing 13 may be composed of a plurality of wires 20 having any desired pattern, configuration, and characteristic. In one such embodiment, the webbing 13 may be analogous to string on a tennis racket. In another embodiment, the webbing 13 may be made from one or more of, for example, stainless steel, nitinol, plastics, or fabrics such as nylon, polyester, cotton, or silk.

The webbing 13 may define a plurality of cells or spaces 19 between the wires 20. The spaces 19 may be of any suitable shape, size, and/or configuration. For example, the spaces 19 may be sized to act as a filter that allows material smaller than a pre-determined size to pass through the filter. In certain embodiments, the size of the spaces 19 may vary depending on the application in which the medical device 1 will be used. For example, the size of the spaces 19 may be in the range of approximately 0.5 mm to approximately 3 mm.

The end-effector loop 14, shown and described in FIGS. 1-7, is an exemplary end-effector loop that may be used in the method and device of the present invention. Any other suitable end-effector loop capable of entrapping, releasing, and/or removing objects from within a body lumen may be used.

As shown in FIGS. 1 and 2, the end-effector loop 14 may be configured to collapse or expand as desired. For example, the end-effector loop 14 may be configured to be collapsible such that it facilitates retraction of some or all of the webbing 13 into lumen 16 of the sheath 12 as shown in FIG. 2, and/or advanced out of the sheath 12, such that the end-effector loop 14 at least partially expands and/or fully expands as shown in FIG. 1. In such an example, the end-effector loop 14 may be collapsed and retracted into lumen 16 of the sheath 12, while the sheath 12 is being advanced through a body lumen to a desired body location. Once the sheath 12 is positioned at the desired body location, the end-effector loop 14 may be advanced out of the sheath 12 for expansion.

The end-effector loop 14 may be retracted into and/or advanced out of lumen 16 of the sheath 12 using any known method in the art. For example, a proximal end of the end-effector loop 14 may be integral with two elongate actuation members 14a, such as wires, that extend through the sheath 12 to an actuation handle 11 for actuation purposes. In other embodiments, a proximal end of the end-effector loop 14 may be attached to the distal end of a single wire strand or cable (not shown) extending from the actuation handle 11, and thereby connecting the end-effector loop 14 to the actuation handle 11. Connection of the end-effector loop 14 to the single wire strand or cable and of the single wire strand or cable to the actuation handle 11 may be accomplished through any suitable means of connection. For example, possible connections may include, but are not limited to, welding, soldering, tying, and/or crimping. Additionally, any suitable known handle assemblies may be used at the proximal user end for actuation. The actuation handle may be made from any suitable material including, but not limited to, thermoplastics, stainless steel, or nitinol. The user may achieve the two basic positions, collapsed (retracted) and expanded (extended), by actuating the actuation handle 11 to move the end-effector loop 14 translationally in the lumen 16 relative to the sheath 12.

In the embodiment of FIG. 1, a connecting member 15 is positioned between the distal end of the end-effector loop 14 and the actuation handle 11. The connecting member 15 may be a flexible material such as suture or wire, or a rigid mechanical linkage, having proximal and distal ends. The connecting member 15 may be provided in the lumen 16 of the sheath 12 and accessible to a user, through actuation handle 11, at the proximal end of lumen 16. The connecting member 15 may be joined at one end to the distal end of the end-effector loop 14 and at the other end to an actuator, for example a knob or other like structure, or any other suitable actuation mechanism. Alternatively, actuator 17 may be provided with the capability of independently actuating both the end-effector loop 14 and the connecting member 15. For example, the actuator 17 may be configured to move distally in slot 18 to actuate the end-effector loop 14, and, in addition, be configured to rotate such that it actuates connecting member 15 for translational movement within sheath 12. The connecting member 15 may be made from any suitable material. Such materials include, but are not limited to, suture, stainless steel, or nitinol. In certain embodiments, the end-effector loop 14 and the distal end of the connecting member 15 may be made from the same material.

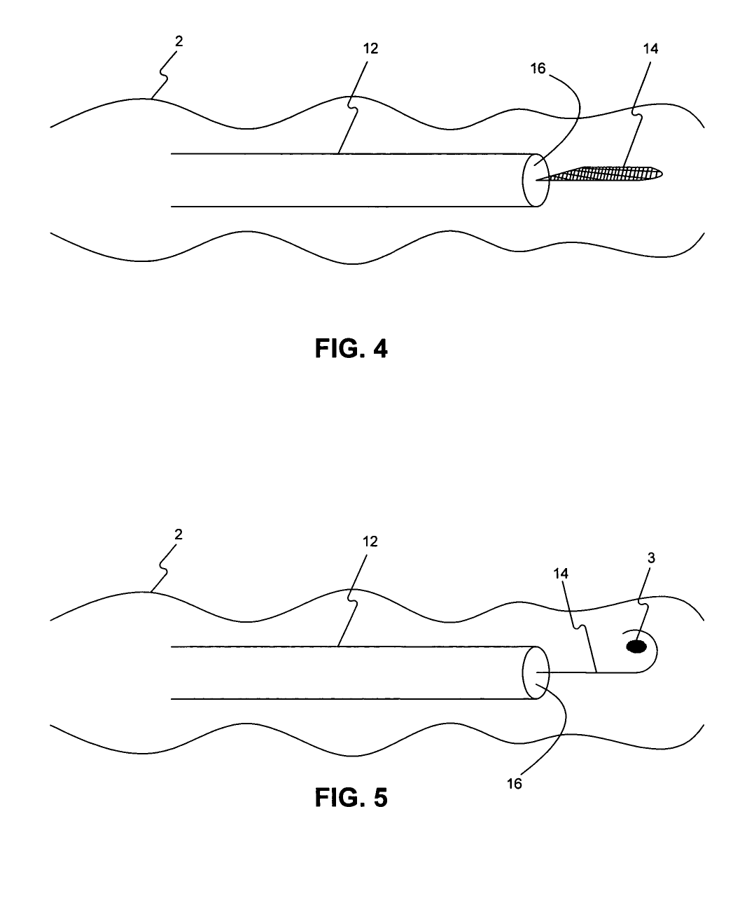

As shown in FIGS. 5-7, the connecting member 15 enables a user to selectively deflect the end-effector loop 14 into various configurations relative to the device. For example, proximal translation of the connecting member 15 may deflect the end-effector loop 14 relative to the longitudinal axis of the device. Distal translation of the connecting member 15 may reduce prior deflection of the end-effector loop 14 relative to the longitudinal axis of the device, or return the end-effector loop to the original position shown in FIG. 4.

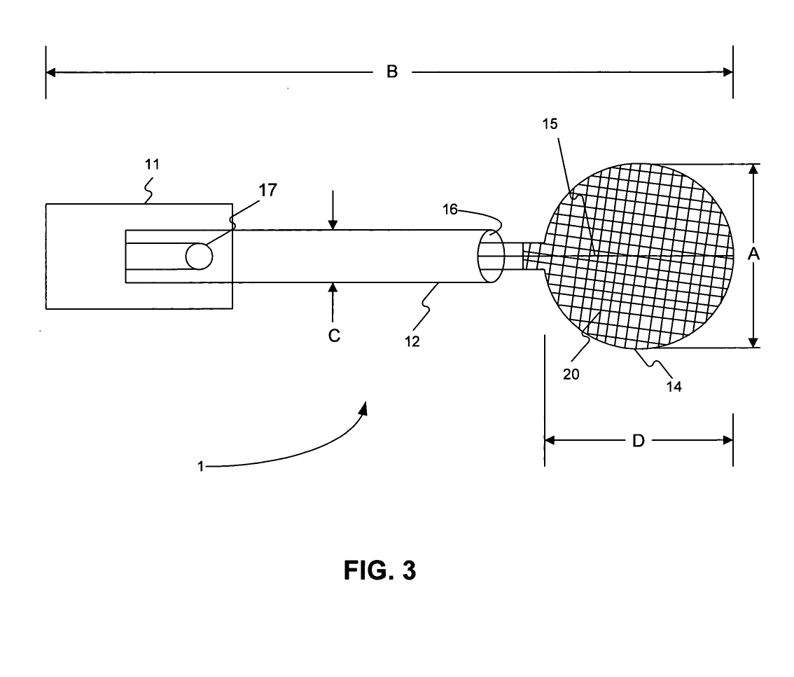

According to an embodiment of the present invention, FIG. 3 is a diagram illustrating exemplary dimensions of medical device 1 in an open position. In one embodiment, the distance A in FIG. 3, from one side of the end-effector loop 14 to the other side of the end-effector loop 14, at its widest point in the fully-extended open position, may be between approximately 0.5 cm to approximately 3 cm inclusive. In another embodiment, the distance B in FIG. 3, from the proximal end of the actuation handle 11 to the fully extended distal end of the end-effector loop 14 in the open position, may be between approximately 20 cm to approximately 200 cm inclusive. In yet another embodiment, the distance C in FIG. 3, the outer diameter of the sheath 12, may be between approximately 1 French to approximately 10 French inclusive. In still another embodiment, the distance D in FIG. 3, from one end of the end-effector loop 14 to the other end of the end-effector loop 14, at its longest point in the fully-extended open position, may be between approximately 1 cm to approximately 3 cm inclusive. These dimensions are exemplary and other suitable dimensions depending, for example, on the desired application, may be used.

In reference to FIGS. 1-7, embodiments of the invention may include methods of using the medical device 1 of the present disclosure to retrieve stones and other unwanted materials located in the bladder, ureter, kidney, or other body structures. Medical device 1 may be used in an environment that is relatively fluid filled or that is relatively dry. The medical device 1 may be inserted through the urethra of a patient, or alternatively, the medical device 1 may be inserted percutaneously. The medical device 1 may be used in any location of the body in which a passageway or orifice includes unwanted material to be removed and/or is being at least partially blocked.

The medical device 1 may be advanced to a treatment site in a number of different ways. For example, the medical device 1 may be advanced to a desired body location with the aid of a guidewire (not shown), wherein the desired body location may correspond to the vicinity of a stone, or another object targeted for removal. The medical device 1 may also be advanced to the treatment site through an access sheath or any other access device known in the art.

Alternatively, the medical device 1 may be fed to the desired body location by means of an imaging device, and may travel through the body without the use of a guidewire or access sheath. To facilitate such feeding, the sheath 12 may include radiopaque marker bands (not shown) detectable by x-ray or other imaging means. Thus, the user may monitor the position of the medical device 1 and movement thereof through the use of an imaging device.

As yet another alternative, the medical device 1 may be used in conjunction with an endoscope (not shown) or any other type of intracorporeal scope known in the art. The endoscope may travel through the body to the treatment site in any conventional manner. Once the endoscope is positioned adjacent to the treatment site, the medical device 1 may be fed through an access port of the endoscope to gain access to the object targeted for removal, such as a stone.

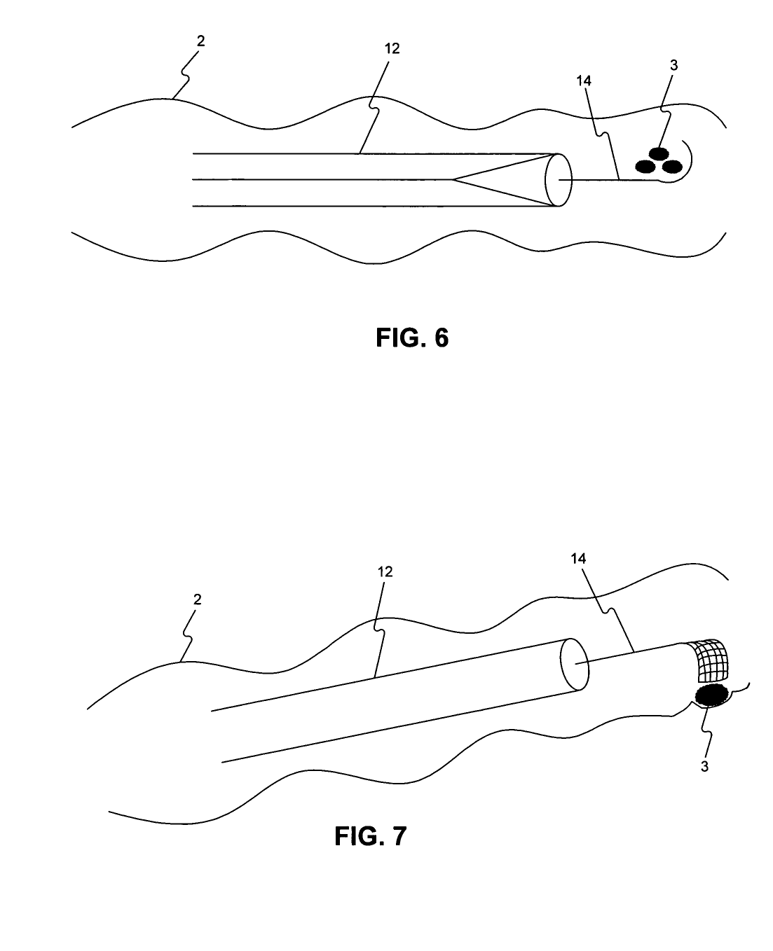

As shown in FIGS. 5-7, the object 3 targeted for retrieval may be a bodily concretion such as, for example, a stone. The object 3 targeted for retrieval may include, but is not limited to, kidney stones, gallbladder stones, uric acid stones, or other solids commonly removed from a body structure or passageway. The object 3 may be of any size and/or shape. Furthermore, the medical device 1 may be used to retrieve objects that are both free-floating, as shown in FIG. 6, or impacted, as shown in FIG. 7.

With the end-effector loop 14 in the collapsed position shown in FIG. 2, a user may insert the distal end of the medical device into an existing anatomical lumen. The medical device 1 then may be advanced through a body lumen to a desired body location. For example, the medical device 1 may be advanced until the distal end of the sheath 12 has reached a desired location, such as, a short distance past an object 3 targeted for entrapment and removal. The method is not limited to use with any particular object, and may also be used with one more intracorporeal and/or extracorporeal objects at various locations and of various geometries and compositions.

Once the distal end of the sheath 12 is advanced past a targeted object, the end-effector loop 14 may then be advanced distally out of the sheath 12, so that the end-effector loop 14 self expands. This may be achieved by, for example, sliding actuator 17 distally in slot 18. When fully deployed, end-effector loop 14 may be substantially circular in shape or have any other configuration suitable to the particular size and shape of both the targeted object 3 and/or the environment it is located in. The proximal end of the end-effector loop 14 may remain in a collapsed state and/or disposed within the sheath 12, and may be the narrowest portion of the end-effector loop 14. Alternatively, the proximal end of the end-effector loop 14 may extend out of the distal end of the sheath 12.

Once the end-effector loop 14 is at least partially deployed, the user may manipulate the end-effector loop 14 to change its position within the tract or lumen. The user may rotate or otherwise change the orientation of the end-effector loop 14 of various embodiments about the axis defined by the sheath 12 by manipulating the actuation handle 11. In addition, the end-effector loop 14 of the medical device 1 is capable of being deflected up to 180 degrees, at degree intervals in the open position, by manipulation of the connecting member 15 by, for example, the actuation handle 11. Thus, the medical device is capable of achieving a variety of positions and, therefore, performing a variety of functions in a patient's body tract or lumen.

In accordance with an embodiment of the present invention, FIG. 5, for example, illustrates the use of a medical device 1 to grasp material within the body. In particular, FIG. 5 illustrates the use of medical device 1 to grasp material in an exemplary anatomical tract 2 of the body. As illustrated in FIG. 5, the end-effector loop 14 of the device is first located in the desired location and orientation within the body, for example, near a stone 3 in the ureter. From the expanded and extended position illustrated in FIG. 4, the distal end of the end-effector loop 14 is then deflected up to 180 degrees around the object(s) 3 to be grasped; thus, capturing the object(s) 3 in the webbing 13 by actuation of the connecting member 15. At this point, the object(s) 3 can be moved, removed from the body, or held stationary within the body tract or lumen. Additionally or alternatively, the object(s) 3 in the webbing may be released from the medical device 1 by manipulating the connecting member 15 to release the distal end of the end-effector loop 14 to return the end-effector loop 14 to the open or expanded position illustrated in FIG. 4.

Alternatively, and as shown in FIG. 6, the medical device 1, in accordance with one embodiment of the present invention, may be used to sweep an anatomical tract 2 occluded with material 3. In particular, FIG. 6 illustrates the use of the end-effector loop 14 of the medical device 1 to sweep material 3 in an exemplary anatomical lumen 2 of the body. The medical device 1 is first located in the desired location and orientation within the body, for example, the end-effector loop 14 of the medical device 1 is positioned near stones 3 occluding, for example, the ureter, as illustrated in FIG. 6. From the expanded and extended position illustrated in FIG. 4, the distal end of the loop is deflected, for example, about 90 degrees; thus, enabling the occluded area to be swept with the end-effector loop 14 and its webbing 13. The user may sweep an occluded area, for example, by maintaining a constant end-effector loop 14 deflection while moving the entire end-effector loop 14 and/or medical device 1 proximally and/or distally.

As yet another alternative, and as shown in FIG. 7, the medical device 1, in accordance with another embodiment of the present invention, may be used to tease or pry material away from an embedded position in an anatomical lumen within the body of a patient. In particular, FIG. 7 illustrates the use of medical device 1 to pry material 3 in an exemplary anatomical tract of the body 2. The medical device 1 is first located in the desired location and orientation within the body, for example, near impacted stones in the ureter, as illustrated in FIG. 7. From the expanded and extended position illustrated in FIG. 4, the distal end of the end-effector loop 14 may be deflected, for example, about 120 degrees; thus, enabling the end-effector loop 14 to pry impacted stones away from the embedded position in the anatomical lumen. The user may pry the material 3 in the body, for example, by manipulating the connecting member 15 to change the deflection of end-effector loop 14 while otherwise keeping the medical device 1 steady.

For example, in various embodiments of the invention, a user can manipulate the actuation handle to control the loop and to pry out lodged or embedded objects to free them from impaction; occlude anatomical tracts and lumens of appropriate inside diameters to prevent migration of foreign objects; sweep the tract or lumen to remove foreign object(s); wrap the loop around a foreign object to move or remove the object; and/or straighten the loop to release it from the foreign object from the loop.

Embodiments of the invention may be used in any medical or non-medical procedure, including any medical procedure where removal of an object from within a body lumen is desired. In addition, at least certain aspects of the aforementioned embodiments may be combined with other aspects of the embodiments, or removed, without departing from the scope of the invention.

Illustrative descriptions of the invention are provided above. However, it is to be understood that the present invention encompasses variations that will be evident to those of ordinary skill in the art from the foregoing descriptions.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A medical device comprising:
a sheath having a proximal end, a distal end, and a lumen extending longitudinally between the proximal and distal ends;
an actuation handle disposed at the proximal end of the sheath;
an end-effector loop adjacent a distal end of the sheath and operably connected to the actuation handle for actuation of the end-effector loop between a deployed position and an undeployed position; and
a connecting member disposed within the lumen of the sheath and connected to the actuation handle and the end-effector loop, wherein actuation of the connecting member deflects the end-effector loop when the end-effector loop is in the deployed position.

2. The device of claim 1, wherein the deployed position is an expanded position and the undeployed position is a collapsed position.

3. The device of claim 2, wherein the end-effector loop is in the collapsed position when retracted into the sheath.

4. The device of claim 1, wherein the end-effector loop is extendable from and retractable within the lumen of the sheath.

5. The device of claim 4, wherein actuation of the connecting member deflects the end-effector loop when the end-effector loop is extended from the lumen of the sheath.

6. The device of claim 1, wherein an elongate actuation member connects the end-effector loop to the actuation handle for actuation of the end-effector loop.

7. The device of claim 6, wherein the elongate actuation member attaches to a proximal end of the end-effector loop, and the connecting member attaches to a distal end of the end-effector loop.

8. The device of claim 6, wherein the elongate actuation member is selected from a group comprising a wire and a cable.

9. The device of claim 1, wherein the connecting member moves within the lumen of the sheath.

10. The device of claim 1, wherein the end-effector loop comprises an outer periphery that defines a space including webbing.

11. The device of claim 10, wherein the webbing includes a plurality of wires.

12. The device of claim 11, wherein the webbing defines a plurality of spaces between the wires.

13. The device of claim 1, wherein the end-effector loop comprises an outer periphery that defines a space including a plurality of wires.

14. The device of claim 1, wherein actuation of the connecting member deflects the end-effector loop up to 180 degrees.

15. A method of entrapping an object within a body comprising:
inserting a medical device into a body lumen, the medical device comprising:
a sheath having a proximal end, a distal end, and a lumen extending longitudinally between the proximal and distal ends;
an actuation handle disposed at the proximal end of the sheath;
an end-effector loop adjacent a distal end of the sheath and operably connected to the actuation handle; and
a connecting member disposed within the lumen of the sheath and connected to the actuation handle and the end-effector loop;
advancing said medical device to a desired body location within a body lumen;
extending said end-effector loop distally relative to the sheath to deploy said end-effector loop into a deployed position;
with the end-effector loop in the deployed position, actuating the connecting member to deflect the end-effector loop; and
entrapping the object with the end-effector loop.

16. The method of claim 15, further comprising the step of removing said medical device and entrapped object from the body.

17. The method of claim 15, wherein the step of deploying the end-effector loop includes expanding the end-effector loop.

18. The method of claim 15, wherein an elongate actuation member operably connects the end-effector loop to the actuation handle.

19. The method of claim 18, wherein the elongate actuation member attaches to a proximal end of the end-effector loop, and the connecting member attaches to a distal end of the end-effector loop.

20. The method of claim 18, wherein the elongate actuation member is selected from a group comprising a wire and a cable.

21. The method of claim 15, wherein the connecting member moves within the lumen of the sheath.

22. The method of claim 15, wherein the end-effector loop comprises an outer periphery that defines a space including webbing.

23. The method of claim 22, wherein the webbing includes a plurality of wires.

24. The method of claim 23, wherein the webbing defines a plurality of spaces between the wires.

25. The method of claim 15, wherein the end-effector loop comprises an outer periphery that defines a space including a plurality of wires.

26. The method of claim 15, wherein the step of actuating the connecting member deflects the end-effector loop up to 180 degrees.

27. The method of claim 15, wherein the end-effector loop is extended distally relative to the sheath from a collapsed position.

28. The method of claim 15, further comprising the step of prying an object from an embedded position within a body.

29. The method of claim 28, wherein the step of prying an object from an embedded position within a body is performed prior to entrapping the object with the end-effector loop.

30. The method of claim 27, wherein the end-effector loop is in the collapsed position when retracted into the lumen of the sheath.
